(19) United States
(12) Patent Application Publication
(10) Pub. No.: US 2004/0242885 A1
(43) Pub. Date: Dec. 2, 2004

(54) Manufacture of vitamin b6

(76) Inventors: Werner Bonrath, Freiburg (DE); Barbara Heller, Dettmannsdorf (DE)

Correspondence Address: Stephen M Haracz, Bryan Cave, 1290 Avenue of the Americas, New York, NY, 10104, US

(21) Appl. No.: 10/490,196

(22) PCT Filed: Sep. 12, 2002
(86) PCT No.: PCT/EP02/10226

(30) Foreign Application Priority Data
Sep. 21, 2001 (EP) 011222499

(51) Int. Cl. C07D 491/02

(52) U.S. Cl. 546/115

(57) ABSTRACT

A process for the manufacture of a=:1-1,3-dihydrofuro[3,4-c]pyridine (I) 1 which is 4,7-disubstituted with a readily cleavable group (R) involves reacting a bis(3-R-substituted 2-propynyl) ether of the formula (R—C≡C—CH₂)₂O (II) with acetonitrile in the presence of a cobalt(I) complex catalyst of the formula XCo(I)Lig_{1/2} (III), wherein X signifies cyclopentadienyl, acetylcyclopentadienyl, indenyl or phenylborinato and Lig_{1/2} signifies the cyclooctadiene or norbornadiene ligand (Lig1) or two carbonyl or ethene ligands (Lig₂), in an aliphatic, alicyclic or aromatic hydrocarbon solvent, or in excess acetonitrile as the reagent and simultaneously the solvent, or in water alone or in admixture with an ethereal co-solvent, or in a mixture of two or more of the aforementioned types of solvents, at a temperature in the range from about 0° C. to about 80° C., under light irradiation at wavelengths from about 300 nm to about 800 nm and with as much exclusion of atmospheric oxygen as possible. The so-produced 4,7-di(R)-substituted 6-methyl-1,3-dihydrofuro[3,4-c]pyridine is useful as an intermediate in the multistage process for manufacturing vitamin B6, especially pyridoxine. The invention further concerns the novel bis(3-R′-substituted 2-propynyl) ethers of the formula (R′—CC≡CH₂)₂O (II′), wherein R′ signifies a tri(C₂₋₈-alkyl)silyl group, and the novel 4,7-di(R′)-substituted 6-methyl-1,3-dihydro-furo[3,4-c]pyridines.

The present invention concerns a process for manufacturing vitamin B₆, especially pyridoxine, and more particularly an intermediate process step in the multistage manufacturing process whereby an intermediate compound, namely a 4,7-disubstituted 6-methyl-1,3-dihydrofuro[3,4-c]pyridine, is produced under certain reaction conditions by the catalysed [2+2+2]-cycloaddition of a bis(3-substituted 2-propynyl) ether with acetonitrile.

The cobalt complex-catalysed [2+2+2]-cycloaddition of bis(3-trimethylsilyl-2-propynyl) ether with acetonitrile to afford 6-methyl-4,7-bis(trimethylsilyl)-1,3-dihydrofuro[3,4-c]pyridine is known from the work of K. Schleich et al., Helv. Chim. Acta 67, 1274-1282 (1984), and K. P. C. Vollhardt et al., Tetrahedron 41, No. 24, 5791-5796 (1985). Schleich et al. used cobaltocen (dicyclopentadienylcobalt, [CoCp₂]) as the catalyst for the [2+2+2]-cycloaddition and conducted the reaction at 145° C. and under a pressure of 100 bar in an autoclave for one hour in a large excess of acetonitrile as the solvent. Using cyclopentadienyl-dicarbonyl-cobalt, [CpCo(CO)₂], as the catalyst Vollhardt et al. carried out the same reaction in deoxygenated m-xylene and excess acetonitrile at reflux temperature, i.e. at about 139° C., while irradiating the reaction mixture with a 250 W slide projector lamp; the total reaction time was about 36 hours. In both cases the reaction conditions are harsh, the reaction temperature being high, a large relative amount of catalyst being required and either a high pressure or a long reaction time being involved. Moreover, Schleich et al. report that a by-product of the reaction is formed by the aromatization of three alkyne groups from two molecules of bis(3-trimethylsilyl-2-propynyl) ether, but that its production can be largely suppressed by using a large excess of acrylonitrile. These represent the disadvantages of the prior art encountered in the process for manufacturing 6-methyl-4,7-bis(trimethylsilyl)-1,3-dihydrofuro[3,4-c]pyridine.

The object of the present invention is the provision of a process for manufacturing 6-methyl-4,7-bis(trimethylsilyl)-1,3-dihydrofuro[3,4-c]pyridine and analogous 4,7-disubstituted 6-methyl-1,3-dihydrofuro[3,4-c]pyridines (the 4- and 7-substituents being readily cleavable groups, preferably trialkylsilyl groups), which process does not have the above-indicated disadvantages of the previously known procedures. This object is substantially achieved by the process of the present invention, which is a process for the manufacture of a 4,7-disubstituted 6-methyl-1,3-dihydrofuro[3,4-c]pyridine of the general formula.

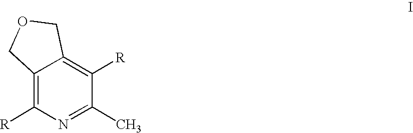

wherein R signifies a readily cleavable group characterized by reacting a bis(3-R-substituted 2-propynyl) ether of the general formula.

(R—C≡C—CH₂)₂O II

wherein R has the significance given above, with acetonitrile in the presence of a cobalt(I) complex catalyst of the general formula

XCo(I)Lig_{1/2} III

wherein X signifies cyclopentadienyl, acetylcyclopentadienyl, indenyl or phenylborinato,

and Lig_{1/2} signifies the cyclooctadiene or norbornadiene ligand (Lig₁) or two carbonyl or ethene ligands (Lig₂),

in an aliphatic, alicyclic or aromatic hydrocarbon solvent, or in excess acetonitrile as the reagent and simultaneously the solvent, or in water alone or in admixture with an ethereal co-solvent, or in a mixture of two or more of the aforementioned types of solvents, at a temperature in the range from about 0° C. to about 80° C., under light irradiation in the wavelength range from about 300 nm to about 800 nm and with as much exclusion of atmospheric oxygen as possible.

In the above-defined compounds of formulae I and II the readily cleavable group R is preferably a tri(C₁₋₈-alkyl)silyl group. Those alkyl groups therein with three or more carbon atoms can be straight-chain or branched. The three alkyl groups can be the same or different, examples of such trialkylsilyl groups being trimethylsilyl, triethylsilyl and tert. butyldimethylsilyl. The group R is preferably trimethylsilyl or tert. butyldimethylsilyl.

The cobalt(I) complexes of the formula III and used as the catalysts in the process of the present invention are in each case either a known compound or can be produced by methods analogous to the published methods for producing the known complexes: see, for example, J. Organomet. Chem. 160, 17-23 (1978) and ibid. 451, 23-31 (1993).

Examples of these cobalt(I) complexes are cyclopentadienyl-cyclooctadienyl-cobalt(I), [CpCo(cod)]; acetylcyclopentadienyl-cyclooctadienyl-cobalt(I), [Cp^{ac}Co(cod)]; indenyl-cyclooctadienyl-cobalt(I), [IndCo(cod)]; phenylborinato-cyclooctadienyl-cobalt(I), [(PhB)Co(cod)]; cyclopentadienyl-biscarbonyl-cobalt(I), [CpCo(CO)₂]; and cyclopentadienyl-bisethene-cobalt(I), [CpCo(C₂H₄)₂], of which [CpCo(cod)] is the preferred cobalt(I) complex catalyst and also commercially available.

The non-polar aprotic hydrocarbon solvent, preferably an aliphatic, alicyclic or aromatic hydrocarbon, which amongst alternative solvents can be used in the process of the present invention, is especially a C₅₋₁₆-alkane, a C₅₋₈-cycloalkane or an optionally alkyl-(especially methyl-) substituted benzene, respectively, of which preferred examples are pentane, hexane and heptane; cyclohexane; and toluene, respectively. Apart from excess acetonitrile or water, a further alternative solvent is an aqueous mixture in which the non-aqueous component, i.e. the co-solvent, is an ethereal solvent, the latter suitably being a lower aliphatic ether or a cyclic ether, e.g. diethyl ether, or dioxan or tetrahydrofuran, respectively. Depending on the nature of such an ethereal co-solvent, this may be water-immiscible, e.g. diethyl ether, or significantly water-miscible, e.g. dioxan or tetrahydrofuran, so that mono- or diphasic solvent systems come into question as the aqueous-ethereal solvents usable in the process according to the present invention. In the case of the other mixed solvents foreseen (“a mixture of two or more of the aforementioned types of solvents”), these may also be mono- or diphasic solvent systems depending on the nature of the components of such systems.

The process is generally effected at temperatures from about 0° C. to about 80° C., preferably from about 20° C. to about 60° C.

Furthermore, the process is generally carried out under light irradiation in the above-mentioned wavelength range from about 300 nm to about 800 nm. Preferably this range is from about 350 nm to about 500 nm. The actual nature of the light source emitting light irradiation in the said wavelength range is immaterial to the success of the process and may be for example a lamp, e.g. a slide projector lamp, or the natural light irradiation means sunlight, of which sunlight, with a wavelength range within the aforementioned broad range, is a preferred irradiation source.

Moreover, the process is conveniently carried out without the need to apply elevated pressure, so that atmospheric pressure conditions are generally suitable.

In order to ensure that as much exclusion of atmospheric oxygen as possible is maintained during the process, this is conveniently carried out under an inert atmosphere, preferably under gaseous nitrogen or argon.

Furthermore, the molar ratio of acetonitrile (if solely used as the one reactant, and not also as solvent) to the bis(3-R-substituted 2-propynyl) ether of the formula II in the reaction mixture is conveniently about 1:1 to about 10:1. If acetonitrile is employed not only as the one reactant but also as the solvent, and thus in relatively large excess based on the amount of diyne of formula II employed, then the molar ratio is conveniently about 50:1 to about 10000:1, preferably about 100:1 to about 1000:1.

The amount of catalyst of formula III used is such that the percentage molar amount of catalyst relative to the employed amount of reactant [bis(3-R-substituted 2-propynyl) ether of formula II or acetonitrile] which is in the lesser molar amount (usually the diyne of formula II) is conveniently about 0.1 to about 2.0 mole %, preferably about 0.5 to about 1.2 mole %.

Conveniently about 0.5 to about 20 ml of solvent (in the case where acrylonitrile itself is not used in excess in the dual role of reactant and solvent) are used per mmol of bis(3-R-substituted 2-propynyl) ether of formula II. Preferably this range is 7-10 ml/mmol, more preferably 3-7 ml/mmol.

The actual reaction generally lasts for about 2 to about 10 hours, preferably about 4 to about 6 hours.

The process in accordance with the invention can be carried out batchwise or continuously, preferably continuously, and in general operationally in a very simple manner involving the addition of the diyne to the acetonitrile and any additional solvent containing the catalyst, or of the acetonitrile to the diyne and any additional solvent containing the catalyst (the one reactant being added to the other in any desired sequence) at room temperature, followed by heating and irradiating the reaction mixture at the desired temperature and irradiation levels under constant stirring.

After completion of the reaction the produced 4,7-disubstituted 6-methyl-1,3-dihydrofuro[3,4-c]pyridine of formula I can be isolated and purified also in a very simple manner, for example by cooling the mixture after completion of the reaction and collecting the resulting crystalline product by filtration, or by initial removal of the solvent and remaining reactants from said mixture by evaporation followed by distillation of the desired product at the appropriate elevated temperature and reduced pressure.

An advantage of the process of the present invention over and above the already-mentioned advantages compared with the state of the art (Schleich et al. and Vollhardt et al.; see above) of a much lower reaction temperature and reaction time, the reduced amount of catalyst required and the avoidance of a high pressure resides in a much more selective synthesis of the desired product of formula I, i.e. considerable suppression of the production of the by-product formed by the aromatization of three alkyne groups from two molecules of the diyne starting material of formula II and of other by-products.

The starting bis(3-R-substituted 2-propynyl) ethers of formula II are, apart from the specific compound bis(3-trimethylsilyl-2-propynyl) ether, novel compounds. Bis(3-trimethylsilyl-2-propynyl) ether itself is known) e.g. from the aforementioned articles of K. Schleich et al. and K. P. C. Vollhardt et al., and can be produced by the methods, i.e. involving a Grignard reaction or a silylation of the butyllithium derivative of di(2-propynyl) ether, respectively, described in these articles. The remaining, novel bis(3-trialkylsilyl-2-propynyl) ethers of formula II can be produced by analogous methods to those for producing the known bis(3-trimethylsilyl-2-propynyl) ether. The novel bis(3-R-substituted 2-propynyl) ethers of formula II, i.e. of formula II′ featuring instead of R the symbol R′, wherein R′ signifies a tri(C₂₋₈-alkyl)silyl group, constitute a further aspect of the present invention.

By the same token, those compounds of formula I wherein R signifies a tri(C₂₋₈-alkyl)silyl group denoted as R′ above, i.e. the compounds of the following general formula

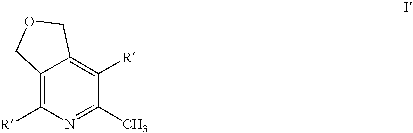

are also novel and constitute a still further aspect of the present invention.

The invention is illustrated by the following Examples:

### Production of the 4,7-disubstituted 6-methyl-1,3-dihydrofuro[3,4-c]pyridines of formula I

### EXAMPLE 1

Into a two-necked reaction flask equipped with a thermostat, a magnetic stirrer and inert gas (argon) gasification were introduced under the argon atmosphere 1 mmol of a bis(3-R-substituted 2-propynyl) ether of formula II (the diyne reactant; e.g. 0.238 g of bis(3-trimethylsilyl-2-propynyl) ether), 1 mmol (0.05 ml) of acetonitrile and 1 ml of the employed solvent containing 0.01 mmol of the catalyst (e.g. 2.3 mg of cyclopentadienyl-cyclooctadienyl-cobalt (I), [CpCo(cod)]). A further 9 ml of solvent were then added, and the reaction mixture was stirred at the thermostatically selected temperature and continuously irradiated with light at a wavelength of about 350 nm emitted by two 460 W Phillips HPM 12 lamps. After reaction time of 6 hours in each case the reaction was established to have gone to completion.

When pentane was used as the solvent the produced 4,7-disubstituted 6-methyl-1,3-dihydrofuro[3,4-c]pyridine of formula I (e.g. 6-methyl-4,7-bis(trimethylsilyl)-1,3-dihydrofuro[3,4-c]pyridine) could either be crystallized out of solution on cooling the mixture in a refrigerator and separated by filtration, or could be isolated by removing the solvent and remaining reactants by distillation of the mixture at the end of the reaction. Using other solvents, e.g. hexane, cyclohexane or toluene) the produced pyridine derivative was isolated by distillation

The varied reaction conditions (catalyst, diyne reactant: significance of R, temperature and solvent,) and the yield of respective product (based on the amount of diyne reactant employed) in each case are given in the following Table:

TABLE 1

| Catalyst | Diyne reactant: R | Temperature | Solvent | Yield |
|---|---|---|---|---|
| [IndCo(cod)] | Si(CH₃)₃ | 35° C. | Pentane | 63% |
| [IndCo(cod)] | Si(CH₃)₃ | 60° C. | Hexane | 66% |
| [Cp^{ac}CO(cod)] | Si(CH₃)₃ | 35° C. | Pentane | 75% |
| [Cp^{ac}Co(cod)] | Si(CH₃)₃ | 25° C. | Pentane | 65% |
| [Cp^{ac}Co(cod)] | Si(CH₃)₃ | 60° C. | Hexane | 75% |
| [CpCo(CO)₂] | Si(CH₃)₃ | 40° C. | Hexane | 19% |
| [CpCo(cod)] | Si(CH₃)₃ | 35° C. | Pentane | 80% |
| [CpCo(cod)] | Si(CH₃)₃ | 60° C. | Hexane | 70% |
| [CpCo(cod)] | Si(CH₃)₃ | 40° C. | Hexane | 73% |
| [CpCo(cod)] | Si(CH₃)₃ | 25° C. | Hexane | 72% |
| [(PhB)Co(cod)] | Si(CH₃)₃ | 40° C. | Toluene | 39% |
| [(PhB)Co(cod)] | Si(CH₃)₃ | 40° C. | Hexane | 70% |
| [CpCo(cod)] | tert. C₄H₉Si(CH₃)₂ | 40° C. | Hexane | 29% |
| [(PhB)Co(cod)] | tert. C₄H₉Si(CH₃)₂ | 40° C. | Hexane | 51% |

Characterization of the produced compounds 6-methyl-4,7-bis(trimethylsilyl)-1,3-dihydrofuro[3,4-c]pyridine (Ia) and 6-methyl-4,7-bis-(tert. butyldimethylsilyl)-1,3-dihydrofuro[3,4-c]pyridine (Ib):

Ia ¹H NMR (400 MHz, CDCl₃): δ 0.35 (s, 9H, Si(CH₃)₃), 0.44 (s, 9H, Si(CH₃)₃), 2.78 (s, 3H, CH₃), 5.31 (s, 4H, CH₂OCH₂); Mass spectroscopy m/z: 279 (M⁺, 12); 264 (29), 251 (25), 237 (27), 236 (100), 73 (49); Elemental analysis for C₁₄H₂₅NOSi₂ (MW 279.53): Calc'd C, 60.16%, H, 9.01%; Found C, 59.75%, H, 8.9%.

Ib ¹H NMR (400 MHz, CDCl₃): δ 0.37 (s, 6H, Si(CH₃)₂), 0.48 (s, 6H, Si(CH₃)₂), 1.27 (s, 9H, C(CH₃)₃), 2.79 (s, 3H, CH₃), 5.30 (s, 4H, CH₂OCH₂); Mass spectoscopy m/z: 363 (M⁺, 6), 348 (11), 307 (100), 290 (23), 251 (37), 222 (82), 131 (8); Elemental analysis for C₂₀H₃₇NOSi₂ (MW 363.68): Calc'd C, 66.05%, H, 10.25%, N, 3.85%; Found C, 65.75%, H, 10.30%, N, 3.99%.

### EXAMPLE 2

Into a two-necked reaction flask equipped with a thermostat, a magnetic stirrer and inert gas (argon) gasification were introduced under the argon atmosphere 0.645 g (2 mmol) of bis(3-tert. butyldimethylsilyl-2-propinyl) ether, 0.1 ml (2 mmol) of acetonitrile and 10 ml of hexane containing 4.6 mg (0.02 mmol) of the catalyst cyclopentadienyl-cyclooctadienyl-cobalt(I), [CpCo(cod)]. The reaction mixture was stirred at the thermostatically selected temperature (40° C.) and continuously irradiated with light at a wavelength of about 350 nm emitted by two 460 W Phillips HPM 12 lamps. After a reaction time of about 6 hours the reaction was established to have gone to completion. 6-Methyl-4,7-bis-(tert.butyldimethylsilyl)-1,3-dihydrofuro[3,4-c]pyridine was separated in 30% yield from the reaction mixture by distillation. The product featured the characterizing data as given for compound Ib at the end of Example 1.

### EXAMPLE 3

Into a two-necked reaction flask equipped with a thermostat, a magnetic stirrer and inert gas (argon) gasification were introduced under the argon atmosphere 0.645 g (2 mmol) of bis(3-tert.butyldimethylsilyl-2-propinyl) ether, 0.1 ml (2 mmol) of acetonitrile and 10 ml of toluene containing 0.02 mmol of the catalyst phenylborinato-cyclooctadienyl-cobalt(I), [(PhB)Co(cod)]. The reaction mixture was stirred at the thermostatically selected temperature (40° C.) and continuously irradiated with light at a wavelength of about 350 nm emitted by two 460 W Phillips HPM 12 lamps. After a reaction time of about 6 hours the reaction was established to have gone to completion. 6-Methyl-4,7-bis-(tert.butyldimethylsilyl)-1,3-dihydrofuro-[3,4-c]pyridine was separated in 50% yield from the reaction mixture by distillation. The product featured the characterizing data as given for compound Ib at the end of Example 1.

### EXAMPLE 4

Into a two-necked reaction flask equipped with a thermostat, a magnetic stirrer and inert gas (argon) gasification were introduced under the argon atmosphere 0.645 g (2 mmol) of bis(3-tert.butyldimethylsilyl-2-propinyl) ether, 0.1 ml (2 mmol) of acetonitrile and 10 ml of cyclohexane containing 0.02 mmol of the catalyst phenylborinato-cyclooctadienyl-cobalt(I), [(PhB)Co(cod)]. The reaction mixture was stirred at the thermostatically selected temperature (40° C.) and continuously irradiated with light at a wavelength of about 350 nm emitted by two 460 W Phillips HPM 12 lamps. After a reaction time of about 6 hours the reaction was established to have gone to completion. 6-methyl-4,7-bis-(tert.butyldimethylsilyl)-1,3-dihydrofuro[3,4-c]pyridine was separated in 70% yield from the reaction mixture by distillation. The product featured the characterizing data as given for compound Ib at the end of Example 1.

### EXAMPLE 5

Into a two-necked reaction flask equipped with a thermostat, a magnetic stirrer and inert gas (argon) gasification were introduced under the argon atmosphere 0.645 g (2 mmol) of bis(3-tert.butyldimethylsilyl-2-propinyl) ether, 0.1 ml (2 mmol) of acetonitrile and 10 ml of hexane containing 0.02 mmol of the catalyst phenylborinato-cyclooctadienyl-cobalt(I), [(PhB)Co(cod)]. The reaction mixture was stirred at the thermostatically selected temperature (40° C.) and continuously irradiated with light at a wavelength of about 350 nm emitted by two 460 W Phillips HPM 12 lamps. After a reaction time of about 6 hours the reaction was established to have gone to completion. 6-Methyl-4,7-bis-(tert.butyldimethylsilyl)-1,3-dihydrofuro[3,4-c]pyridine was separated in 70% yield from the reaction mixture by distillation. The product featured the characterizing data as given for compound Ib at the end of Example 1.

### Production of the bis(3-R-substituted 2-propynyl) ether reactant of formula II

### EXAMPLE 6

In a 100 ml three-necked flask equipped with a magnetic stirrer, dropping funnel and reflux condenser were placed 2.066 g (85 mmol) of magnesium shavings and 9 ml of tetrahydrofuran. To this mixture were then added 6.48 ml (87 mmol) of freshly distilled ethyl bromide in 9 ml of tetrahydrofuran. The reaction mixture was stirred for one hour at reflux temperature.

Then 4.36 ml (42.5 mmol) of dipropargyl ether in 25 ml of tetrahydrofuran were added over a period of one hour, maintaining the temperature at a maximum of 40° C. with stirring followed by 13 g (0.86 mol) of tert. butyldimethylchlorosilane in 18 ml of tetrahydrofuran, added dropwise to the reaction mixture at 40° C. within a further hour. The mixture was stirred for a further hour at 40 ° C. and then cooled down to room temperature. After addition of 15.7 ml of water an ochre coloured suspension resulted. The separated aqueous phase was washed with 12 ml of tetrahydrofuran, and the combined organic phases were washed with saturated aqueous sodium chloride solution and dried over a molecular sieve. After removal of the organic solvent by evaporation the residue was distilled at 91° C./0.034 mbar (3.4 Pa). Bis(3-tert.butyldimethylsilyl-2-propinyl) ether was isolated in about 80% yield.

¹H NMR (400 MHz, CDCl₃): δ 0.1030 (s, 12H, 2×(CH₃)₂Si), 0.9286 (s, 18H, 2×C(CH₃)₃), 4.2592 (s, 4H, 2×CH₂O); ¹³C NMR (400 MHz, CDCl₃): δ −4.7250 (CH₃Si), 16.4503 (C(CH₃)), 26.0238 (C(CH₃)), 57,0190 (OCH₂O), 90.3080 (C≡CSi), 101.1516 (CH₂—C≡C); Mass Spectroscopy m/z: 322 (M⁺, 1), 265 (23), 235 (20), 209 (34), 155 (63), 73 (100); Elemental Analysis for C₁₈H₃₄OSi₂ (MW 322.63): Calc'd C, 67.01%, H, 10.62%; Found C, 66.85%, H, 10.700%.

1. A process for the manufacture of a 4,7-disubstituted 6-methyl-1,3-dihydrofuro[3,4-c]pyridine of the general formula 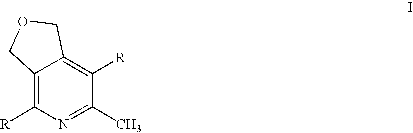
wherein R signifies a readily cleavable group, characterized by reacting a bis(3-R-substituted 2-propynyl) ether of the general formula.
(R—C≡C—CH₂)₂O II
wherein R has the significance given above, with acetonitrile in the presence of a cobalt(I) complex catalyst of the general formula
XCo(I)Lig_{1/2} III
wherein X signifies cyclopentadienyl, acetylcyclopentadienyl, indenyl or phenylborinato,
and Lig_{1/2} signifies the cyclooctadiene or norbornadiene ligand (Lig₁) or two ethene ligands (Lig₂),
in an aliphatic, alicyclic or aromatic hydrocarbon solvent, or in excess acetonitrile as the reagent and simultaneously the solvent, or in water alone or in admixture with an ethereal co-solvent, or in a mixture of two or more of the aforementioned types of solvents, at a temperature in the range from about 0° C. to about 80° C., under light irradiation in the wavelength range from about 300 nm to about 800 nm and with as much exclusion of atmospheric oxygen as possible, the percentage molar amount of catalyst of formula III relative to the employed amount of reactant bis(3-R-substituted 2-propynyl) ether of formula II or acetonitrile which is in the lesser molar amount being about 0.1 to about 2.0 mole %.

2. A process according to claim 1, wherein the readily cleavable group R is a tri(C₁₋₈-alkyl)silyl group.

3. A process according to claim 1, wherein the cobalt(I) complex of the formula III is cyclopentadienyl-cyclooctadienyl-cobalt(I), acetylcyclopentadienyl-cyclooctadienyl-cobalt(I), indenyl-cyclooctadienyl-cobalt(I), phenylborinato-cyclooctadienyl-cobalt(I) or cyclopentadienyl-bisethene-cobalt(I).

4. A process according to claim 1, wherein the aliphatic, alicyclic or aromatic hydrocarbon solvent is a C₅₋₁₆-alkane, a C₅₋₈-cycloalkane or an optionally alkyl-(especially methyl-) substituted benzene, respectively.

5. A process according to claim 1, wherein the process is effected at temperatures from about 20° C. to about 60° C.

6. A process according to claim 1, wherein the process is effected in the wavelength range from about 350 nm to about 500 nm.

7. A process according to claim 1, wherein acetonitrile is not used as both reactant and solvent, and the molar ratio of acetonitrile to the bis(3-R-substituted 2-propynyl) ether of the formula II in the reaction mixture is about 1:1 to about 10:1.

8. A process according to claim 1, wherein the percentage molar amount of catalyst of formula III relative to the employed amount of reactant bis(3-R-substituted 2-propynyl) ether of formula II or acetonitrile which is in the lesser molar amount is about 0.5 to about 1.2 mole %.

9. A process according to claim 1, wherein acetonitrile is not used as both reactant and solvent, and the amount of solvent used per mmol of bis(3-R-substituted 2-propynyl) ether of formula II is about 0.5 to about 20 ml/mmol.

10-11. (Canceled).

12. A process according to claim 2, wherein the readily cleavable group R is trimethylsilyl or tert. butyldimethylsilyl.

13. A process according to claim 3, wherein the cobalt(I) complex of the formula III is cyclopentadienyl-cyclooctadienyl-cobalt(I).

14. A process according to claim 14, wherein the aliphatic, alicyclic, or aromatic hydrocarbon solvent is pentane, hexane, or heptane; cyclohexane; or toluene, respectively.

15. A process according to claim 1, wherein the non-aqueous component (ethereal co-solvent) of the water and ethereal co-solvent mixture is a lower aliphatic ether or a cyclic ether.

16. A process according to claim 15, wherein the lower aliphatic ether is diethyl ether and the cyclic ether is dioxan or tetrahydrofuran.

17. A process according to claim 1 wherein acetonitrile is employed not only as the one reactant but also as the solvent, and the molar ratio of acetonitrile to the bis(3-R-substituted 2-propynyl) ether of the formula II in the reaction mixture is about 50:1 to about 10000:1.

18. A process according to claim 17, wherein the molar ration is about 100:1 to about 1000:1.

19. A process according to claim 9, wherein the amount of solvent used per mmol of bis(3-R-substituted 2-propynyl) ether of formula II is about 7-10 ml/mmol.

20. A process according to claim 19, wherein the amount of solvent used per mmol of bis(3-R-substituted 2-propynyl) ether of formula II is about 3-7 ml/mmol.
